(19) United States
(12) Patent Application Publication
(10) Pub. No.: US 2023/0190161 A1
(43) Pub. Date: Jun. 22, 2023

(54) METHOD AND SYSTEM FOR DETECTING URINATION USING WEARABLE DEVICE

(71) Applicant: RIM HEALTHCARE CO., LTD., Seoul (KR)

(72) Inventor: Gyehwan KIM, Incheon (KR)

(21) Appl. No.: 17/905,398

(22) PCT Filed: Mar. 4, 2021
(86) PCT No.: PCT/KR2021/002688

§ 371 Date: Aug. 31, 2022

(30) Foreign Application Priority Data
Mar. 5, 2020 (KR) 10-2020-0027911

(51) Int. Cl. A61B 5/20; G06T 7/00; G06T 7/90; A61B 5/00; G16H 40/67; G16H 50/30

(52) U.S. Cl. CPC A61B 5/20; A61B 5/0059; A61B 5/725; A61B 5/6889; A61B 5/7221; G06T 7/0012; G06T 7/90; G16H 40/67; G16H 50/30

(57) ABSTRACT

A method for detecting urination includes (a) obtaining sensor data S, which is continuous data according to the passage of time and generated by the movement of a measuring device; and (b) filtering the sensor data S as valid data related to urination among the sensor data S obtained in (a) by using a valid data filtering module of the measuring device according to a preset method. The filtering includes filtering the sensor data S obtained when a distance between a sensor installed in a space provided with a urinal or a toilet seat and the measuring device is within a preset distance, as the valid data.

### TECHNICAL FIELD

The present disclosure relates to a method and system for detecting urination using a wearable device.

### BACKGROUND ART

Disorders related to urination include dysuria, urinary retention, delayed urination, frequent urination, nocturia, and drip after urination. In particular, delayed urination symptoms and drip symptoms after urination are commonly found in elderly people, and the cause thereof may be cystitis, and especially in women, the cause may be acute urinary retention, etc., and thus there are various causes.

However, it is difficult to check delayed urination symptoms or drip symptoms after urination unless a patient reveals it himself/herself. In general, it is common to check through a questionnaire with an existing patient or to use a urination diary, which is a urine checklist written by the patient himself/herself.

However, writing a urination diary is cumbersome from the patient’s point of view, and it is difficult to confirm that the check is done correctly.

Thus, in order to resolve such inconvenience, demand has been increasing for a system capable of determining urination information and urination disorders using the urination information by detecting a urination pattern using a wearable device that is always mounted on the body.

The related prior art is as follows.

Korean Patent Registration No. 10-2018416 discloses a method of detecting whether an object to be measured is urinating and ejaculating using a wearable device. However, the method relates to detecting urination or ejaculation is performed by comparing a urination pattern or ejaculation pattern obtained through machine learning with data obtained by the wearable device, and there is a problem in that a large number of data for comparison needs to be accumulated.

Korean Patent Publication No. 10-2019-0066407 discloses a method of detecting urination of an object to be measured using a wearable device. However, there is a problem in that only urination of the object to be measured seated in a toilet seat is detected and urination of the object to be measured using a urinal cannot be detected.

Korean Patent Registration No. 10-2072467 discloses that a user’s urine and feces image information are obtained using a camera, and the user’s health condition is analyzed according to color, turbidity, shape, and amount. However, the health condition is not analyzed by reflecting the user’s personal information except for information that can be obtained from urine and feces, and thus, the accuracy of the analysis is lowered.
(Patent document 1) Korean Patent Registration No. 10-2018416 (Sep. 5, 2019)
(Patent document 2) Korean Patent Publication No. 10-2019-0066407 (Jun. 13, 2019)
(Patent document 3) Korean Patent Registration No. 10-2072467 (Feb. 3, 2020)

### DETAILED DESCRIPTION OF THE INVENTION

### Technical Problem

The present disclosure has been devised to solve the above problems.

Specifically, the present disclosure provides a method and system, whereby urination-related data with high accuracy regardless of the gender of an object to be measured in which a measuring device is mounted, a urinal or a toilet seat, may be collected, and urination information and urination disorder may be determined from the collected data.

In addition, the present disclosure also provides a method and system, whereby health information of an object to be measured may be calculated by photographing a urine image and more accurate health information may be calculated by using various information instead of using only an image.

In addition, the present disclosure also provides a program stored in a computer-readable recording medium to execute the above-described method.

In addition, the present disclosure also provides a computer-readable recording medium in which a computer program for executing the above-described method is recorded.

### Technical Solution

According to an aspect of the present disclosure, there is provided a method of detecting urination, the method including (a) obtaining sensor data S, which is continuous data according to the passage of time and generated by the movement of a measuring device 200; and (b) filtering the sensor data S as valid data related to urination among the sensor data S obtained in (a) by using a valid data filtering module 240 of the measuring device 200 according to a preset method, wherein the filtering includes filtering the sensor data S obtained when a distance between a sensor 300 installed in a space provided with a urinal or a toilet seat and the measuring device 200 is within a preset distance, as the valid data.

(b) may include filtering the sensor data S obtained in (a) as the valid data by using the valid data filtering module 240 when the sensor data S obtained in (a) includes both first sensor data S1 obtained when the measuring device 200 moves for a preset time or more and second sensor data S2 obtained when the measuring device 200 stops for a preset time or more after the first sensor data S1 is obtained.

The method may further include calculating a distance between the measuring device 200 and the sensor 300 by using a distance calculation module 230, and (b) may include filtering the sensor data S as the valid data by using the valid data filtering module 240 when the distance calculated by the distance calculation module 230 is calculated within the preset distance before the second sensor data S3 is obtained after the first sensor data S1 is obtained.

(b) may include filtering the sensor data S as the valid data by using the valid data filtering module 240 when the sensor data S includes third sensor data S3 obtained when the measuring device 200 moves from a preset first height h1 to a second height h2 lower than the first height h1 before the second sensor data S2 is obtained after the first sensor data S1 is obtained, and when the sensor data S includes fourth sensor data S4 obtained when the measuring device 200 moves from the second height h2 to the first height h1 after the second sensor data S2 is obtained.

The second height h2 may be a height corresponding to a knee height of an object to be measured to which the measuring device 200 is mounted.

The sensor 300 may be further installed in the urinal installed in the space, and the distance calculation module 230 may further include calculating the distance between the measuring device 200 and the sensor 300 installed in the urinal, and (b) may include filtering the sensor data S as the valid data by using the valid data filtering module 240 when a state in which the distance calculated by the distance calculation module 230 is within a preset distance, is maintained for a preset time before the second sensor data S2 is obtained after the first sensor data S1 is obtained.

(b) may include filtering the sensor data S as the valid data by using the valid data filtering module 240 when the sensor data S includes fifth sensor data S5 obtained when the measuring device 200 vibrates for a preset time or more with a predetermined amplitude or more after the second sensor data S2 is obtained.

(b) may include filtering the sensor data S as the valid data by using the valid data filtering module 240 when the sensor data S includes sixth sensor data S6 obtained when the measuring device 200 moves from a preset first height h1 to a third height h3 lower than the first height h1 before the second sensor data S2 is obtained after the first sensor data S1 is obtained, and when the sensor data S includes seventh sensor data S7 obtained when the measuring device 200 moves from the third height h3 to the first height h1 after the fifth sensor data S5 is obtained.

The method may further include (c) calculating data at an initial time point at which the measuring device 200 stops, among the second sensor data S2 of the filtered valid data, as a urination start time and calculating data at a time when the measuring device 200 moves after the urination start time, as a urination end time by using a urination information calculation module 250.

The method may further include: (d) calculating a difference between the urination end time and the urination start time as a urination time, and calculating a urination amount according to a preset method using the calculated urination time by using the urination information calculation module 250; and (e) determining whether there is a urination disorder according to a preset method using the urination time and the urination amount calculated in (d), by using the urination disorder determination module 260.

A gender and age of the object to be measured may be input through an information input module 120, and (d) may further include calculating the urination amount in different ways according to the input gender and age, respectively, by using the urination information calculation module 250.

At least one of age, smoking, and pain of the object to be measured may be input through the information input module 120, and the method may further include: (f) obtaining an image by photographing an area containing urine by using a photographing module 110; and (g) calculating health information for the image by using information of at least one of the inputted age, smoking, and pain during urination and color information of the image captured in (c) by using an image calculation module 130.

According to another aspect of the present disclosure, there is provided an apparatus for performing the above-described method.

According to another aspect of the present disclosure, there is provided a system for performing the above-described method, the system including: the measuring device 200 that is mounted on an object to be measured and includes a gyro sensor 221 and an acceleration sensor 222 to generate sensor data S corresponding to the movement, and filters the sensor data S as the valid data related to urination according to a preset method; and the sensor 300 installed in a space provided with the urinal or the toilet seat.

The system may further include a user interface 100 to which at least one of gender, age, smoking status, and pain of the object to be measured is input through the information input module 120 and in which an area containing urine is photographed and which calculates health information for the image by using information of one or more of the input age, smoking status, and pain and color information of the captured image.

According to another aspect of the present disclosure, there is provided a computer program stored in a computer-readable recording medium to execute the above-described method.

According to another aspect of the present disclosure, there is provided a computer-readable recording medium on which the computer program for executing the above-described method is recorded.

### Effects of the Invention

According to the present disclosure described above, the following effects are achieved.

First, urination-related data can be obtained with high accuracy regardless of the gender of an object to be measured and a urinal or a toilet seat.

Second, because only urination-related data among the sensor data obtained by a sensor is filtered as valid data, the reliability of urination information calculated using the filtered valid data that is actually urination-related data is improved.

Third, a user can accurately know his or her urination data without being forced to an excessively inconvenient or complicated procedure, which can help diagnose symptoms and treatment.

Fourth, even from the point of view of a medical staff, the patient’s daily urination data can be accurately transmitted, and the symptoms of delayed urine can be determined based on the received data based on a universal standard, so that accurate determination can be made regardless of skill level.

Fifth, these data can also be recorded on an EMR server of a hospital, so that the patient can comfortably live at home and check all of the data in real time at the hospital, and these data can be accumulated and shared to secure big data useful for medical technology.

Sixth, health information of the object to be measured can be calculated by capturing a urine image, but more accurate health information can be calculated by using a variety of information instead of using only the image.

### DESCRIPTION OF THE DRAWINGS

FIG. 1 is a schematic block diagram for explaining a system for detecting urination according to an embodiment of the present disclosure;

FIG. 2 is a schematic diagram for explaining a space in which urination of an object to be measured is performed;

FIG. 3 is a block diagram for explaining the system for detecting urination of FIG. 1 in more detail;

FIG. 4 is a flowchart illustrating a method of detecting urination according to an embodiment of the present disclosure;

FIG. 5 is a flowchart illustrating a method of detecting urination according to another embodiment of the present disclosure;

FIG. 6 is a flowchart illustrating a method for detecting urination according to another embodiment of the present disclosure;

FIGS. 7 and 8 are diagrams for explaining a process in which urination is performed by an object to be measured, which is a female;

FIGS. 9 and 10 are diagrams for explaining a process in which urination is performed by an object to be measured, which is a male;

FIG. 11 is a diagram for explaining sensor data used for valid data filtering;

FIG. 12 is a diagram for explaining the average amount of urination according to gender and age;

FIG. 13 is a diagram for explaining a state in which urination is detected by a measuring device;

FIG. 14 illustrates an aspect of sensor data obtained by a sensor unit of the measuring device;

FIG. 15 is a diagram for explaining information that can be input through an information input module in an application executed by an app execution module of a user device;

FIG. 16 shows an execution screen of the application of FIG. 15;

FIGS. 17 and 18 are diagrams for explaining a process of calculating health information of a captured image by using an image captured through a photographing module of a user device and information input through an information input module; and

FIG. 19 is a diagram for explaining various types of health information that can be calculated according to color information of pixels included in the captured image.

### MODE OF THE INVENTION

In some cases, well-known structures and devices may be omitted or shown in the form of a block diagram focusing on key functions of each structure and device in order to avoid obscuring the concept of the present disclosure.

Throughout the specification, when a part is said to be “comprising or including” a certain component, it does not exclude other components unless otherwise stated, meaning that other components may be further included. In addition, terms such as ”...unit”, “...group”, and “module” described in the present specification mean a unit that processes at least one function or operation, which may be implemented as hardware or software or a combination of hardware and software. Also, “a or an”, “one”, “the”, and similar related terms in the context of describing the present disclosure (in particular, in the context of the following claims) may be used in a sense including both the singular and the plural unless indicated or clearly contradicted by the context.

In describing the embodiments of the present disclosure, if it is determined that a detailed description of a well-known function or configuration may unnecessarily obscure the gist of the present disclosure, the detailed description thereof will be omitted. In addition, the terms to be described later are terms defined in consideration of functions in an embodiment of the present disclosure, which may vary according to the intentions or customs of users and operators. Therefore, the definition should be made based on the content throughout the present specification.

Hereinafter, the present disclosure will be described in detail with reference to the accompanying drawings.

Hereinafter, it will be noted that the term “system” refers to an object built according to the present disclosure, not a method.

Hereinafter, the term “EMR” refers to an electronic medical record, and a member that processes related information is referred to as an “EMR server”.

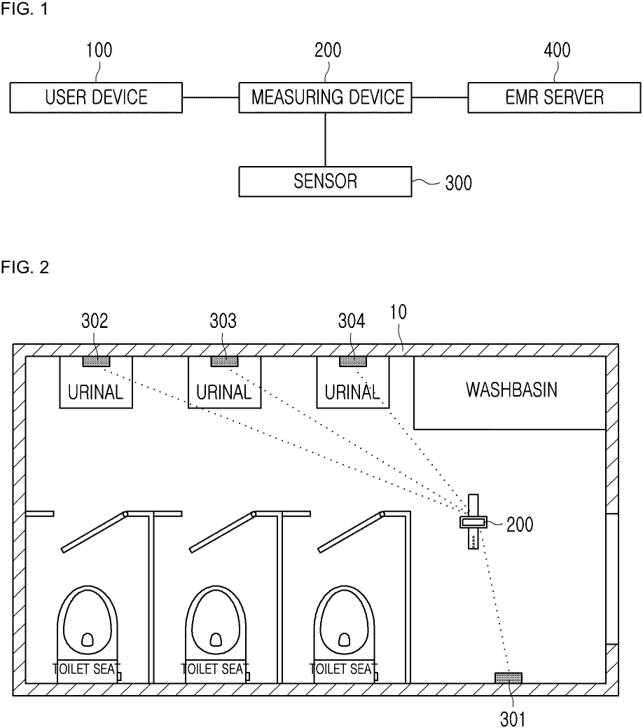

First, referring to FIG. 1, a system for detecting urination according to an embodiment of the present disclosure may include a user device 100, a measuring device 200, a sensor 300, and an EMR server 400.

The user device 100 is a portable and easily movable electronic device that can be carried by an object to be measured may be a videophone, a mobile phone, a smart phone, a portable computer, or a digital camera.

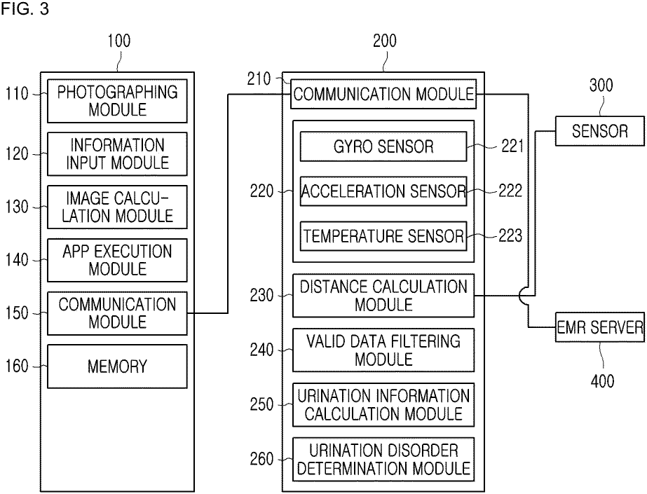

Referring to FIG. 3, the user device 100 may include a photographing module 110, an information input module 120, an image calculation module 130, an app execution module 140, a communication module 150, and a memory 160.

The photographing module 110 may photograph an area facing the user device 100. As an example, a camera may be applied, and photographing may be performed for a urinal or a toilet seat. Preferably, photographing is performed in a state in which urine or feces of the object to be measured are accommodated in the urinal or toilet seat.

The information input module 120 is a part to which information of an object to be measured is input, and the name, gender (female or male), age, smoking status, and pain during urination of the object to be measured may be input. The information input to the information input module 120 may be later used for health information calculation by the image calculation module 130 and urination information calculation by the urination information calculation module 250.

The image calculation module 130 calculates health information for the image captured by the photographing module 110.

A process of calculating health information for a captured image will be described in more detail with reference to FIGS. 17 through 19.

According to an embodiment of the present disclosure, a urine color measurement application may be executed by the app execution module 140 (FIG. 17A), and when the application is executed, a screen is output on which the name, age, and smoking status of the object to be measured may be input (FIG. 17B). On the output screen, information on the name, age, and whether smoking is input to the information input module 120, and photographing by the photographing module 110 may be started. Information input to the information input module 120 may be modified at any time, and the input information is used in all of a process of calculating a urine color, a process of providing health improvement information based on the calculated urine color, and a process of determining a urination amount/urination disorder by urination detection.

In a photographing mode, a box may be displayed in a predetermined area (FIG. 17C), and it is preferable that photographing be performed so that urine or feces are included in the displayed box.

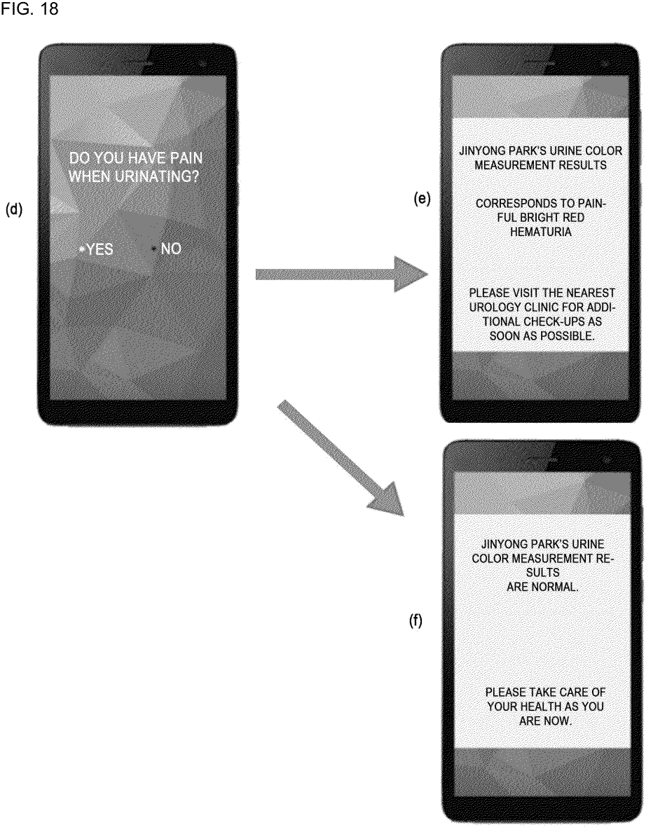

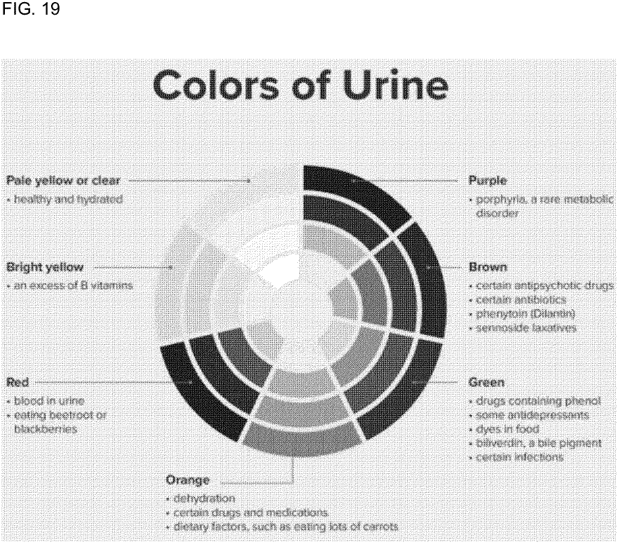

When photographing is performed by the photographing module 110, a screen for checking whether there is pain during urination is output (FIG. 18D), and whether there is pain during urination on the output screen may be input to the information input module 120.

When photographing is completed by the photographing module 110, health information for the captured image of the image calculation module 130 is calculated. More specifically, health information of the image may be calculated using color information of pixels included in the image. In one example, when the user of the user device 100 selects a portion that needs to be captured (e.g., a portion of a toilet seat into which urine is poured) in an image capturing process by using the photographing module 110, the color of the selected area may be calculated based on the color of the user’s urine.

FIG. 19 shows various types of health information that may be calculated according to the color of urine. It is largely divided into 6 colors such as orange, green, brown, purple, bright yellow, and pale yellow, and different colors are shown depending on the brightness in each color.

The image calculation module 130 may calculate health information corresponding to the most similar color by comparing the color information of pixels included in the image with the color shown in FIG. 19. For example, when it is determined that orange is the most similar color, health information may be calculated based on the presence of dehydration symptoms.

In addition, when it is determined by the image calculation module 130 to be hematuria (that is, when red is determined as the most similar color), the result as shown in FIG. 18E may be output to the screen of the user device 100, and when it is determined to be normal, the result as shown in FIG. 18F may be output.

The image calculation module 130 does not simply calculate the health information using only the color information of the pixels included in the captured image, but calculates the health information by further using the information input to the information input module 120. That is, the health information is calculated by further using information on gender, age, smoking status, and pain during urination.

This is because the health information may be determined differently even in urine of the same color depending on gender/age/smoking/pain during urination, and this enables more precise calculation of health information.

In the present disclosure, a variety of health improvement information may be provided through the user device 100 according to the health information calculated by the image calculation module 130. Here, the health improvement information collectively refers to all information for which health information may be improved when a user performs daily life according to information provided by the user, such as food information, exercise information, and life pattern information.

Even if the same health information is calculated according to information such as gender, age, smoking status, pain during urination, etc. input to the user device 100, provided health improvement information may be different. That is, the present disclosure has the advantage that a user-customized health care provision service is possible by providing the most suitable health improvement information to the user according to information such as gender/age/smoking/pain during urination.

The communication module 150 performs communication with the measuring device 200 and the EMR server 400.

An image captured by the photographing module 110, information input to the information input module 120, health information calculated by the image calculation module 130, urination information to be described later, information on whether there is a urination disorder, etc. are stored in the memory 160.

The measuring device 200, as a part mounted on the object to be measured, is mounted on the wrist of the object to be measured, such as a smart watch, a smart band, or a smart ring, and generates sensor data S corresponding to the movement of the object to be measured. However, the measuring device 200 may be mounted not only on the wrist but also on any body part (e.g., the ankle) and is not limited to any measuring device that is attached to clothes, etc. and capable of generating sensor data S corresponding to the movement of the object to be measured. In addition, a unique identifier may be stored, and the unique identifier may be a unique indicator that varies depending on the object to be measured on which the measuring device 200 is mounted.

Referring to FIG. 3, the measuring device 200 may include a communication module 210, a sensor unit 220, a distance calculation module 230, a valid data filtering module 240, a urination information calculation module 250, and a urination disorder determination module 260.

The communication module 210 performs communication with the user device 100 and the EMR server 400.

The sensor unit 220 may output sensor data S corresponding to the movement of the measuring device 200 and may include a gyro sensor 221, an acceleration sensor 222, and a temperature sensor 223. In another embodiment, the sensor unit 220 may further include an atmospheric pressure sensor (not shown).

The gyro sensor 221 and the acceleration sensor 222 output sensor data for 3-axis rotation, etc., and the atmospheric pressure sensor (not shown) detects atmospheric pressure to output sensor data about a height at which the measuring device 200 is spaced apart from the ground (atmospheric pressure decreases as the height increases so that sensor data for the height may be output), and the temperature sensor 223 outputs temperature data about the temperature of the object to be measured in which the measuring device 200 is mounted.

Because the sensor unit 220 outputs continuous sensor data S according to the passage of time and temperature data, the data includes both data related to urination as well as data generated in daily life irrelevant to urination.

Thus, a process of filtering only data related to urination is required. Hereinafter, sensor data used to filter valid data among the sensor data S will be described in detail.

Before this, a space 10 in which the detection of urination is made will be described with reference to FIG. 2.

The space 10 means a space in which a urinal and a toilet seat are installed, and because urination and defecation are generally performed in the space 10 in which the urinal and toilet seat are installed, preferably only the sensor data S generated when the measuring device 200 is located in the space 10 may be filtered as valid data.

To this end, a plurality of sensors 301, 302, 303, and 304 may be installed in the space 10, and the distance calculating module 230 may calculate a distance between the measuring device 200 and the sensors 301, 302, 303, and 304.

In other words, when the distance between the measuring device 200 and the plurality of sensors 301, 302, 303, and 304 calculated by the distance calculation module 230 is within a preset distance, it may be determined that the measuring device 200 is located within the space 10, and the sensor data S obtained when the distance between the measuring device 200 and the sensors 301, 302, 303, and 304 is greater than the preset distance may be determined as noise. As a method of calculating the distance, a method using infrared rays, a triangulation method using at least three or more sensors, etc. may be applied. However, embodiments of the present disclosure are not particularly limited thereto when there is a method, whereby the distance between the sensors 301, 302, 303, 304 and the measuring device 200 may be calculated.

In another embodiment, the number of steps is calculated by a predetermined method using the gyro sensor 221 and the acceleration sensor 222, and when the calculated number of steps is greater than or equal to the predetermined number of steps, it may be determined that the measuring device 200 moves to the space 10, and the sensor data S obtained at this time may be determined as valid data. In this case, the calculated number of steps may be output through the user device 100.

The object to be measured may be a male or a female, and a urination pattern is different depending on the gender.

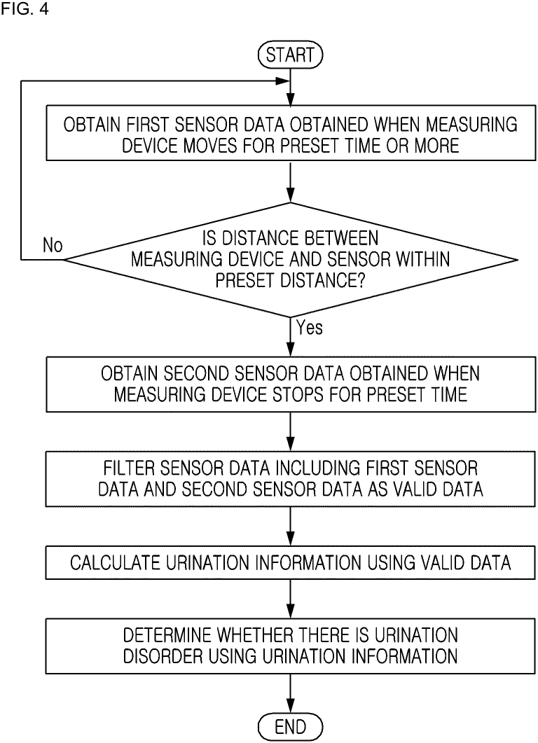

First, a process of filtering valid data based on a urination pattern commonly observed in men and women will be described in detail with reference to FIG. 4.

The object to be measured will move to the space 10 to go to the toilet. Thus, first sensor data S1 generated when the measuring device 200 mounted on the object to be measured moves for a preset time or more is obtained.

Next, the distance between the measuring device 200 calculated by the distance calculation module 230 and the sensors 301, 302, 303, and 304 provided in the space 10 will be calculated within a preset distance, and when the calculated distance is greater than the preset distance, it means that the object to be measured is moving to a place other than the space 10, so that it is determined as noise.

A process of relaxing the muscles or sphincter around the urethra is required to go to the toilet. Thus, the movement of the object to be measured is not observed for the duration of toileting. Because the measuring device 200 stops without movement, second sensor data S2 generated when the measuring device 200 stops for a preset time or more is obtained. Here, the preset time may be 5 to 9 seconds.

The valid data filtering module 240 may filter the sensor data S including the first sensor data S1 and the second sensor data S2 as valid data, and it may be determined whether urination information is calculated by the information calculation module 250 and whether there is a urination disorder by the urination disorder determination module 260 by using the filtered valid data.

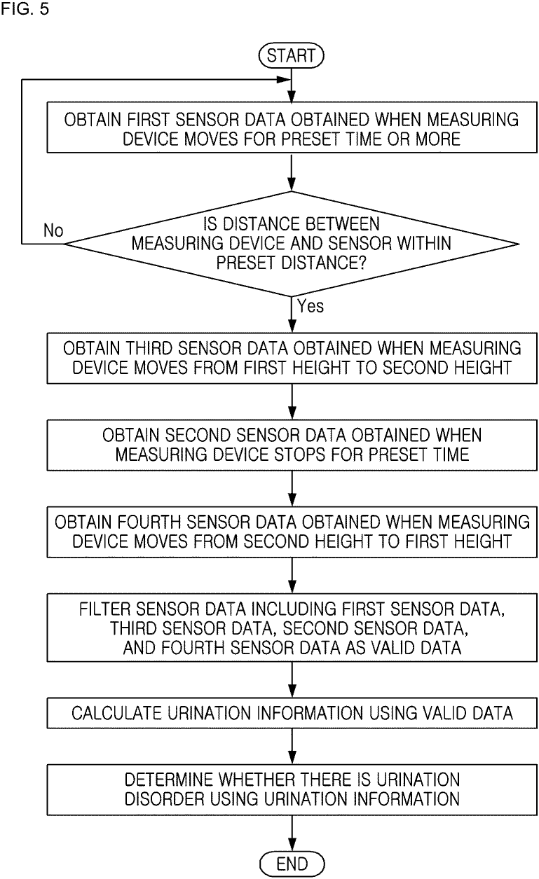

Next, a process of filtering valid data based on the urination pattern of an object to be measured using a toilet seat will be described in detail with reference to FIG. 5.

The object to be measured will move to the space 10 to go to the toilet. Thus, the first sensor data S1 generated when the measuring device 200 mounted on the object to be measured moves for a preset time or more is obtained.

Next, the distance between the measuring device 200 calculated by the distance calculation module 230 and the sensors 301, 302, 303, and 304 provided in the space 10 will be calculated as a value within a preset distance. When the calculated distance is greater than the preset distance, this means that the object to be measured is moving to a place other than the space 10, and thus it is determined as noise.

In order to use the toilet in the toilet seat, a process of taking off the bottom and sitting on the seat of the toilet is involved.

When seated on the toilet seat, a height, which is a distance at which the measuring device 200 is spaced apart from the ground, is lower than when standing. Assuming that the height before seating the toilet seat is a first height h1, when seated in the toilet seat, the measuring device 200 is positioned at a second height h2 that is lower than the first height h1. That is, third sensor data S3, generated when the measuring device 200 moves from the first height h1 to the second height h2 when seated on the toilet seat, is obtained. In order to not only sit down, but also to take off the bottom, the hand is required to be positioned from a waist level to a knee level below a knee height, so that the change from the first height h1 to the second height h2 may be considered as a process of taking off the bottom.

Here, preferably, the second height h2 is a height from the ground to the knee of the object to be measured. As a result of the analysis of urination posture, there were many cases of toileting after putting hands on the thighs. In this case, because the measuring device 200 worn on the object to be measured is also located at the same height as the knee height, the sensor data S corresponding to the second height h2 will be observed.

In addition, because the skin is exposed to the outside when the pants are taken off, the body temperature decreases, and because even after urinating, the body temperature decreases, physiologically, a temporary rise in body temperature due to muscle tremors or contractions occurs in order to prevent a decrease in body temperature. The temperature sensor 223 may output temperature data that is the temperature of the object to be measured, and may filter the data as valid data when the temperature data includes data increasing in numerical value.

Next, the second sensor data S2 generated when the measuring device 200 stops for a preset time or more is obtained.

Next, the object to be measured puts on the bottom and stands up in the sitting posture. In this case, the height of the measuring device 200 will be located again at the first height h1, and fourth sensor data S4 generated when the measuring device 200 moves from the second height h2 to the first height h1 is obtained.

The valid data filtering module 240 may filter the sensor data S including the first sensor data S1, the third sensor data S3, the second sensor data S2, and the fourth sensor data S4 as valid data. Urination information calculation by the urination information calculation module 250 and whether there is a urination disorder by the urination disorder determination module 260 may be determined using the filtered valid data.

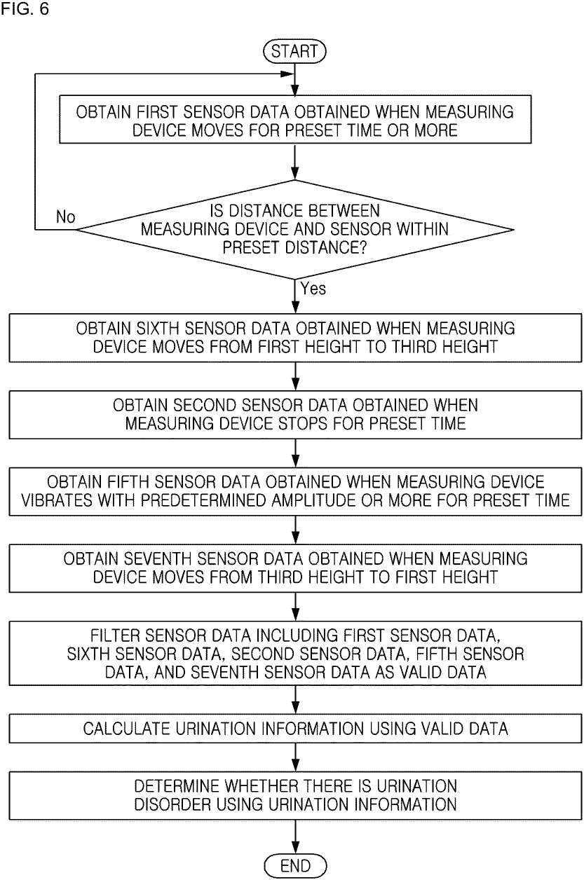

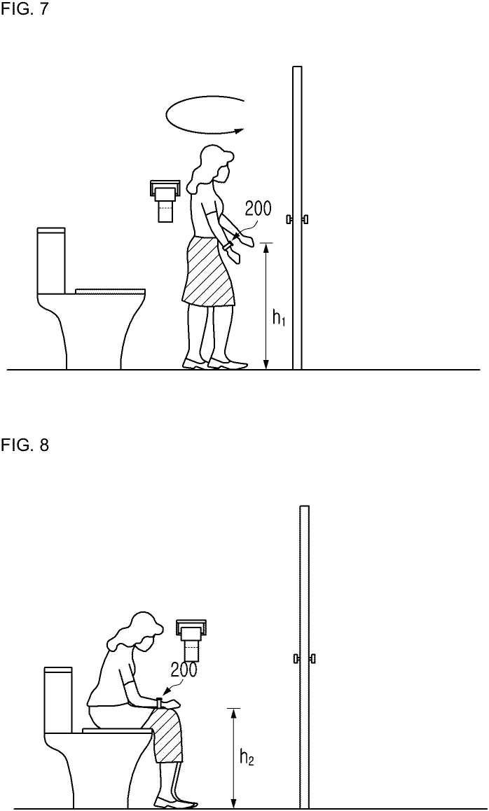

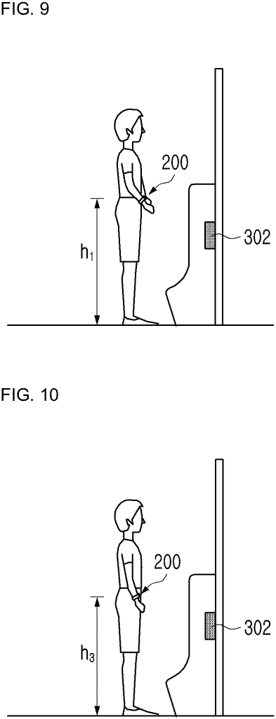

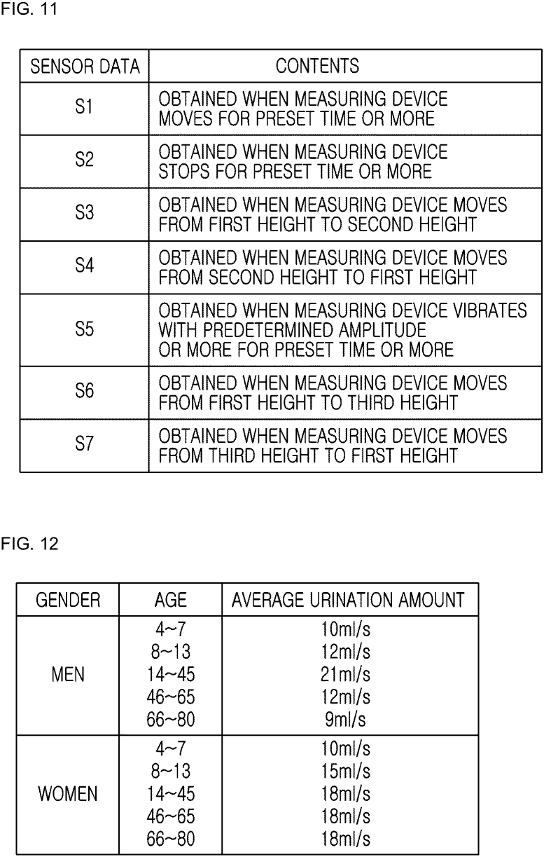

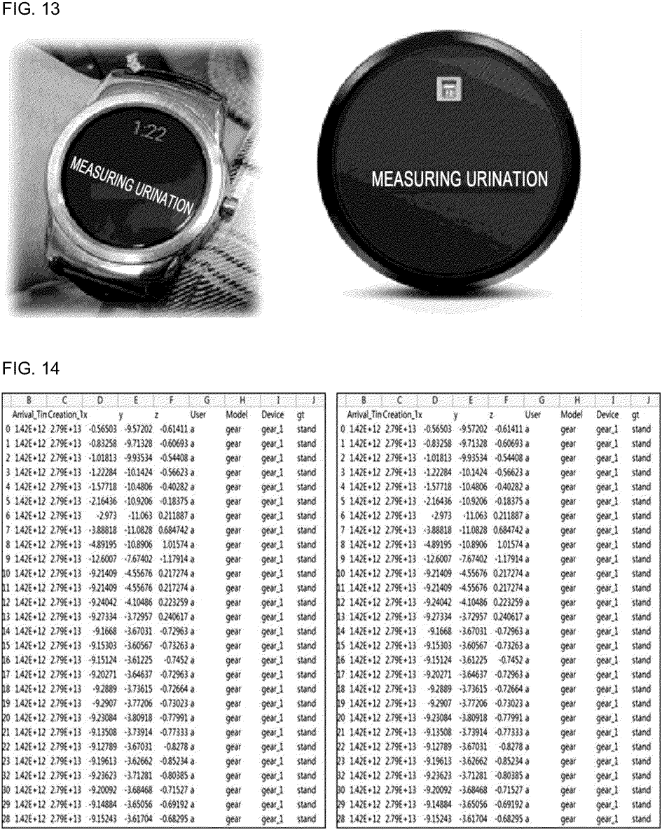

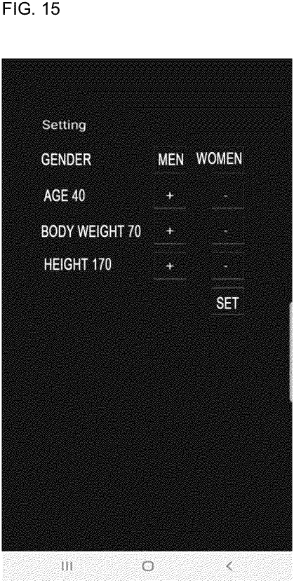

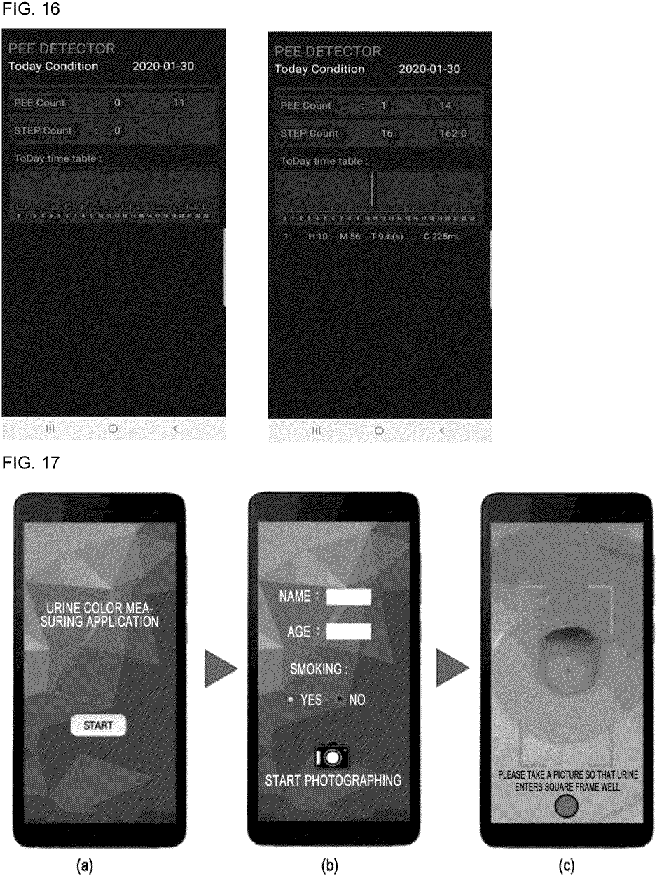

Next, a process of filtering valid data based on a urination pattern of an object to be measured using a urinal will be described in detail with reference to FIG. 6.

In the case of the detecting method illustrated in FIG. 5, the urination of the object to be measured who defecates in the toilet seat may be detected, but it is difficult to detect the urination of the object to be measured who defecates in the urinal. This is because the process of sitting on the toilet seat is performed when toileting in the toilet, but the above-described process is not performed when toileting in the urinal. Thus, a method capable of filtering as valid data even when urinating in a urinal is required.

The object to be measured will move to the space 10 to go to the toilet. Thus, the first sensor data S1 generated when the measuring device 200 mounted on the object to be measured moves for a preset time or more is obtained.

Next, the distance between the measuring device 200 calculated by the distance calculation module 230 and the sensors 301, 302, 303, and 304 provided in the space 10 will be calculated as a value within a preset distance. In particular, the distance between the sensors 302, 303, and 304 provided in each of the urinals and the measuring device 200 may be calculated as a value within a preset distance. The distance between the sensors 302, 303, and 304 and the measuring device 200 may be determined as valid data because the object to be measured may be considered as being positioned in front of the urinal to urinate in the urinal, and when the distance between the sensors 302, 303, and 304 and the measuring device 200 is greater than the preset distance, this means that the object to be measured is moving to a place other than the space 10, so that it is determined as noise.

A process in which a male object to be measured unzips her pants to urinate or partially takes off her pants is accompanied. The measuring device 200 will be moved from the first height h1 to a third height h3 lower than the first height h1, and sixth sensor data S6 generated when the measuring device 200 moves from the first height h1 to the third height h3 is obtained. Here, the third height h3 may be lower than the first height h1 but higher than the second height h2.

Next, the second sensor data S2 generated when the measuring device 200 stops for a preset time or more is obtained.

After the male object to be measured urinates, the act of popping the genitals is performed to expel residual urine. The measuring device 200 will vibrate for a preset time or more with a predetermined amplitude or more, and fifth sensor data S5 generated when the measuring device 200 vibrates with a predetermined amplitude or more for a preset time or more is obtained.

A process in which an object to be measured who completes urination, zips his or her pants or puts on his pants is accompanied, and seventh sensor data S7 generated when the measuring device 200 moves from the third height h3 to the first height h1 is obtained.

The valid data filtering module 240 may filter the sensor data S including the first sensor data S1, the sixth sensor data S6, the second sensor data S2, the fifth sensor data S5, and the seventh sensor data S7 as valid data, and urination information calculation by the urination information calculation module 250, and whether there is a urination disorder by the urination disorder determination module 260 may be determined using the filtered valid data.

That is, the valid data filtering module 240 determines whether the sensor data S is valid data related to urination through a combination of the first sensor data through the seventh sensor data, and data in which the numerical value of the temperature data increases.

Even when only one data of the above data is included, the valid data filtering module 240 may filter it as valid data, and when both data are included, the valid data filtering module 240 may filter them as valid data, and only when all data is included, the valid data filtering module 240 may filter them as valid data. It is obvious that the accuracy of filtering improves as the number of items that are required to include data in order to filter as valid data increases.

It is significant that the data used for filtering are required to be obtained within a preset time. Because the sensor data S is continuous data according to the passage of time, the above data is required to be simultaneously obtained for a preset time to be considered valid data related to urination.

On the other hand, there are cases in which a user seated on a toilet seat not only urinates but also feces. Because the objective of the present disclosure is to detect urine of a user of the measuring device 200, data determined to be defecating may be determined as noise.

In general, in the case of feces, there is a high possibility of sitting on the toilet seat for a longer time than when urinating, and a large amount of toilet paper is used compared to urine even after urinating.

Based on the above difference, in the present disclosure, when a state in which the measuring device 200 is spaced apart from the floor by the second height h2 is maintained for a predetermined urination time or more, or when the measuring device 200 has a predetermined amplitude for using of the toilet paper and the sensor data that vibrates is observed a predetermined number of times or more, it may be determined that the user of the measuring device 200 has defecated.

The predetermined urination time is a very long time compared to the standard urination time, and may be, for example, 2 minutes. In addition, the predetermined number of times may be, for example, 3 to 5 times, but embodiments of the present disclosure are not particularly limited thereto.

The urination information calculation module 250 calculates data at an initial time point at which the measuring device 200 stops, among the second sensor data S2 included in the valid data filtered by the valid data filtering module 240, as a urination start time t1, and calculates data at a time point at which the measuring device 200 moves after the urination start time t1, as a urination end time t2. In the present disclosure, the time corresponding to the urination start time t1 or the urination end time t2 is determined as a urination time, and information on which urination was performed during 24 hours a day according to the urination time may be output through the user device 200. In addition, information on the time at which the user urinated based on one week, one month, and one year according to the determined urination time may also be output through the user device 200, so that regularity information, nocturia information, etc. may be further calculated using this information.

Also, the difference between the urination end time t2 and the urination start time t1 may be calculated as the urination time, and a urination amount may be calculated using the urination time. Here, the urination amount may be calculated by multiplying the urination time by the average urination amount, and more specifically, as shown in FIG. 10, the urination amount may be calculated by multiplying the calculated urination time by an average urination amount value corresponding to the gender and age input to the information input module 120. The calculated urination amount is mapped together with the corresponding urination time and output through the user device 100. This urination amount-urination time may be output in the form of a graph, and the user’s urination pattern may be more conveniently checked.

The urination disorder determination module 260 is a part that determines whether there is a urination disorder by using the urination time and the urination amount calculated by the urination information calculation module 250.

Specifically, the urination disorder determination module 260 may diagnose a delayed urine symptom when the urination time is greater than a preset time. Here, the preset time may be 30 seconds.

As will be described later, the urination disorder determination module 260 may access the EMR server 400 to determine whether there is a urination disorder.

The EMR server 400 is a part capable of receiving the sensor data S, a unique identifier, and the calculated urination time and urination amount from the measuring device 200.

The EMR server 400 may be any conventionally widely used EMR server. However, the received data can be accurately recorded and maintained only when the unique identifier received from the measuring device 200 is stored.

The urination disorder determination module 260 of the measuring device 200 may access the EMR server 400 and determine whether there is a urination disorder using the urination time and amount stored in the EMR server 400, and as a result of the determination, the urination time and the urination amount may also be output to the user device 100.

The method for detecting urination according to an embodiment of the present disclosure described above may be implemented in the form of a program command that can be executed through various computer means and recorded in a computer-readable medium. The computer-readable medium may include program instructions, data files, data structures, etc. alone or in combination. The program instructions recorded on the medium may be specially designed and configured for the present disclosure, or may be known and available to those skilled in the art of computer software. Examples of the computer-readable recording medium include magnetic media such as hard disks, floppy disks and magnetic tapes, optical media such as CD-ROMs and DVDs, magneto-optical media such as floppy disks, and hardware devices specially configured to store and execute program instructions, such as ROM, RAM, flash memory, and the like. Examples of program instructions include not only machine language codes such as those generated by a compiler, but also high-level language codes that can be executed by a computer using an interpreter or the like. The hardware devices described above may be configured to operate as one or more software modules to carry out the operations of the present disclosure, and vice versa.

While the present disclosure has been particularly shown and described with reference to exemplary embodiments thereof, it will be understood by those of ordinary skill in the art that various changes in form and details may be made therein without departing from the spirit and scope of the present disclosure as defined by the following claims.

### EXPLANATION OF REFERENCE NUMERALS

10: space
100: user device
110: photographing module
120: information input module
130: image calculation module
140: app execution module
150: communication module
160: memory
200: measuring device
210: communication module
220: sensor unit
221: gyro sensor
222: acceleration sensor
223: temperature sensor
230: distance calculation module
240: valid data filtering module
250: urination information calculation module
260: urination order determination module
300: sensor
400: EMR server

1-17. (canceled)

18. A method, comprising:
obtaining sensor data S of an object to be measured, the obtained sensor data S being continuous data according to a passage of time and generated by a movement of a measuring device; and
filtering the sensor data S as valid data related to urination among the obtained sensor data S by using a valid data filtering module of the measuring device according to a preset method,
wherein the filtering step comprises filtering the obtained sensor data S when a distance between a sensor installed in a space provided with a urinal or a toilet seat and the measuring device is within a preset distance, as the valid data.

19. The method of claim 18, wherein the filtering step comprises filtering the obtained sensor data S as the valid data by using the valid data filtering module when the obtained sensor data S comprises both first sensor data S1 obtained when the measuring device moves for a preset time or more and second sensor data S2 obtained when the measuring device stops for a preset time or more after the first sensor data S1 is obtained.

20. The method of claim 19, further comprising:
calculating a distance between the measuring device and the sensor by using a distance calculation module,
wherein the filtering step comprises filtering the obtained sensor data S as the valid data by using the valid data filtering module when the distance calculated by the distance calculation module is calculated within the preset distance before the second sensor data S2 is obtained after the first sensor data S1 is obtained.

21. The method of claim 20, wherein the filtering step comprises filtering the obtained sensor data S as the valid data by using the valid data filtering module when the sensor data S comprises third sensor data S3 obtained when the measuring device moves from a preset first height h1 to a second height h2 lower than the first height h1 before the second sensor data S2 is obtained after the first sensor data S1 is obtained, and when the sensor data S comprises fourth sensor data S4 obtained when the measuring device moves from the second height h2 to the first height h1 after the second sensor data S2 is obtained.

22. The method of claim 21, wherein the second height h2 is a height corresponding to a knee height of the object to which the measuring device is mounted.

23. The method of claim 20, wherein the sensor is further installed in the urinal installed in the space, the distance calculation module calculating the distance between the measuring device and the sensor installed in the urinal, and wherein the filtering step comprises filtering the obtained sensor data S as the valid data by using the valid data filtering module when a state in which the distance calculated by the distance calculation module is within a preset distance, is maintained for a preset time before the second sensor data S2 is obtained after the first sensor data S1 is obtained.

24. The method of claim 23, wherein the filtering step comprises filtering the obtained sensor data S as the valid data by using the valid data filtering module when the sensor data S comprises fifth sensor data S5 obtained when the measuring device vibrates for a preset time or more with a predetermined amplitude or more after the second sensor data S2 is obtained.

25. The method of claim 24, wherein the filtering step comprises filtering the obtained sensor data S as the valid data by using the valid data filtering module when the sensor data S comprises sixth sensor data S6 obtained when the measuring device moves from a preset first height h1 to a third height h3 lower than the first height h1 before the second sensor data S2 is obtained after the first sensor data S1 is obtained, and when the sensor data S comprises seventh sensor data S7 obtained when the measuring device moves from the third height h3 to the first height h1 after the fifth sensor data S5 is obtained.

26. The method of claim 19, further comprising:
calculating data at an initial time point at which the measuring device stops, among the second sensor data S2 of the filtered valid data, as a urination start time; and
calculating data at a time when the measuring device moves after the urination start time, as a urination end time by using a urination information calculation module.

27. The method of claim 26, further comprising:
calculating a difference between the urination end time and the urination start time as a urination time;
calculating a urination amount according to a preset method using the calculated urination time by using the urination information calculation module; and
determining whether there is a urination disorder according to a preset method using the urination time and the urination amount calculated above, by using a urination disorder determination module.

28. The method of claim 27, wherein a gender and age of the object are input through an information input module, and wherein the calculating steps further comprise calculating the urination amount in different ways according to the gender and the age, respectively, by using the urination information calculation module.

29. The method according to claim 18, wherein at least one of age, smoking, and pain of the object is input through an information input module, and wherein the method further comprising:
obtaining an image by photographing an area containing urine by using a photographing module; and
calculating health information for the image by using information of at least one of the inputted age, smoking, and pain during urination and color information of the image captured in the calculating by using an image calculation module.

30. An apparatus for performing steps of a method of claim 18.

31. A system, comprising:
a measuring device mounted on an object to be measured, the measuring device including a gyro sensor and an acceleration sensor; and
a sensor installed in a space provided with a urinal or a toilet seat,
wherein sensor data S is obtained, the obtained sensor data S being continuous data according to a passage of time and generated by a movement of the measuring device,
wherein the obtained sensor data S is filtered as valid data related to urination among the sensor data S by using a valid data filtering module of the measuring device according to a preset method, and
wherein the filtered sensor data S is obtained when a distance between the sensor and the measuring device is within a preset distance, as the valid data.

32. The system of claim 31, further comprising:
a user interface configured to receive input of the object through an information input module, the input including at least one of gender, age, smoking status, and pain, wherein an area containing urine is captured and wherein health information for an image is calculated by using information of one or more of the inputted age, smoking status, and pain and color information of the captured image.

33. A computer program stored in a computer-readable recording medium to execute the steps of method of claim 18.

34. A computer-readable recording medium on which a computer program for executing the method of claim 18 is recorded.
